(19) United States
(12) Patent Application Publication
(10) Pub. No.: US 2026/0083985 A1
(43) Pub. Date: Mar. 26, 2026

(54) METHODS FOR TREATMENT PLANNING

(71) Applicant: Elekta, Inc., Atlanta, GA (US)

(72) Inventors: Rui Lopes, Brossard (CA); Martin Emile Lachaine, Montreal (CA)

(21) Appl. No.: 19/338,087

(22) Filed: Sep. 24, 2025

(30) Foreign Application Priority Data
Sep. 24, 2024 (GB) 2414021.2

(51) Int. Cl. A61N 5/10

(52) U.S. Cl. CPC A61N 5/1037

(57) ABSTRACT

A computer-implemented method is disclosed for generating a radiation treatment plan for a patient for at least one future radiation treatment session. The method comprises obtaining one or more images of the patient. The method further comprises, generating, based on the one or more images, a representation of a plurality of potential anatomical states of the patient which may occur during the future radiation treatment session. The method further comprises, generating, based on the representation, at least one treatment plan. Such a method can reduce treatment margins and can improve the adaptive radiotherapy process by improving speed, safety and reduce the need for physician presence.

### CLAIM FOR PRIORITY

This application claims the benefit of priority of British Application No. 2414021.2, filed Sep. 24, 2024, which is hereby incorporated by reference in its entirety.

### FIELD OF THE INVENTION

The present disclosure relates to methods for treatment planning. More specifically, the present invention relates to a computer-implemented method for radiation treatment planning, and data processing apparatuses, computer programs, and non-transitory computer-readable storage mediums configured to execute methods for radiation treatment planning.

### BACKGROUND OF THE INVENTION

Radiation therapy or radiotherapy may be described as the use of ionising radiation to damage or destroy unhealthy cells in both humans and animals. The ionising radiation may be directed to tumours on the surface of the skin or deep inside the body. Common forms of ionising radiation include X-rays and charged particles. An example of a radiotherapy technique is Gamma Knife®, where a patient is irradiated using a number of lower-intensity gamma rays that converge with higher intensity and high precision at a targeted region (e.g., a tumour). Another example of radiotherapy comprises using a linear accelerator (“linac”), whereby a targeted region is irradiated by high-energy particles (e.g., electrons, high-energy photons, and the like). In another example, radiotherapy may be provided using a heavy charged particle accelerator (e.g., protons, carbon ions, and the like).

The placement and dose of the radiation beam may be accurately controlled to provide a prescribed dose of radiation to the target region (e.g., the tumour) and to reduce damage to surrounding healthy tissue (known as organs at risk or OARs). An aspect of treatment planning concerns determining suitable characteristics of radiation to be delivered to produce a safe and effective dose. Characteristics of radiation relate to, for example, a fluence pattern or distribution. The fluence pattern may be dependent on beam arrangements, energies, and field sizes, which are in turn related to controllable parameters (which are optimizable). By determining suitable values for those parameters, a suitable fluence pattern may be obtained. A treatment plan may be determined by a treatment planning system.

A radiation therapy treatment plan (treatment plan, or simply plan) may be established using an optimization procedure to determine a set of optimum parameter values or optimum variable values that are expected to deliver a suitable dose. The optimization procedure may be based on clinical and dosimetric objectives and constraints. Examples of clinical and dosimetric objectives and constraints include maximum, minimum, and mean doses to target regions and surrounding regions (e.g., tumours and critical organs). Clinical and dosimetric objectives and constraints may be referred to as treatment planning objectives. Optimization is usually carried out with respect to one or more treatment plan parameters to reduce beam-on time, improve dose uniformity, etc.

Radiotherapy treatment plans are typically based on a 2D or 3D reference image of the patient, e.g. a CT scan. The reference image is used to identify a target region and to identify critical organs near the target region. The target region (or area to be treated, e.g., a planned target volume, PTV), and surrounding region (or Organs at Risk, OARs) may be contoured by a physician, optionally assisted by automatic segmentation algorithms. After segmentation, a dose plan may be created for the patient indicating the desirable amount of radiation to be received by the target region and/or the surrounding region. The target region may have an irregular volume and may be distinctive in terms of its size, shape, and position. The goal is to deliver a prescribed dose to the target, while ensuring the dose to OARs remain below critical threshold values. There are many potential trade-offs that can be made in achieving a clinically acceptable dose distribution, and often multiple solutions are explored before the physician settles on one that is deemed the most appropriate for the patient.

Therefore, it is desirable to provide an improved radiotherapy treatment planning workflow.

### SUMMARY OF THE INVENTION

It is an aim of the present disclosure to at least partially address one or more of the challenges mentioned above. The invention is defined in the independent claims, to which reference should now be made. Further features are set out in the dependent claims.

According to a first aspect of the present disclosure, there is provided a computer-implemented method for generating a radiation treatment plan for a patient for at least one future radiation treatment session, the method comprising: obtaining one or more images of the patient; generating, based on the one or more images, a representation of a plurality of potential anatomical states of the patient which may occur during the future radiation treatment session; and generating, based on the representation, at least one treatment plan.

In some embodiments, the generating of the representation comprises generating one or more translations and one or more rotations of the one or more images. For example, this could involve generating a plurality of translations and rotations of the one or more images.

In some embodiments, the generating of the representation comprises modelling deformation vector fields, DVFs, e.g. using b-splines and/or optical flow algorithms and/or biomechanical models.

In some embodiments, the generating of the representation uses a network trained on data from multiple patients, the data from each patient comprising a plurality of images of the patient, acquired at different times.

In some embodiments, the network comprises one or more of:
[symbol] a recurrent neural network, and preferably a long short-term memory, LTSM, recurrent neural network or a gated recurrent unit, GRU, recurrent neural network;
[symbol] a variational autoencoder, VAE, and preferably a temporal VAE or a recurrent VAE;
[symbol] a generative adversarial network, GAN;
[symbol] a sequence-to-sequence model with an attention mechanism;
[symbol] a transformer model;
[symbol] a vision transformer model;
[symbol] a temporal transformer model;
[symbol] a physics-informed neural network, PINN.

In some embodiments, the representation of the plurality of potential anatomical states of the patient comprises one or more of the following:
[symbol] a plurality of reference images, wherein each reference image represents one of the plurality of potential anatomical states;
[symbol] a plurality of encoded reference images, wherein each encoded reference image represents one of the plurality of potential anatomical states;
[symbol] a plurality of deformation vector fields, DVFs, wherein each DVF deforms the one or more images of the patient to a reference image, wherein each reference image represents one of the plurality of potential anatomical states;
[symbol] a plurality of encoded DVFs, wherein each encoded DVF deforms the one or more images of the patient to a reference image, wherein each reference image represents one of the plurality of potential anatomical states;
[symbol] a probabilistic distribution of a plurality of reference images, wherein each reference image represents one of the plurality of potential anatomical states, and preferably wherein the probabilistic distribution comprises a probability density function (PDF) representing a likelihood of the patient being in any one of the potential anatomical states.

In some embodiments, the one or more images of the patient comprise one or more of the following:
[symbol] one or more images obtained via measurement of the patient;
[symbol] one or more images obtained via simulation;
[symbol] one or more CT scans of the patient;
[symbol] one or more 4D CT scans of the patient;
[symbol] one or more MRI scans of the patient;
[symbol] one or more 4D MRI scans of the patient;
[symbol] one or more historical images of the patient.

In some embodiments, the method further comprises reducing the dimensionality of the representation, prior to generating the at least one treatment plan.

In some embodiments, reducing the dimensionality of the representation is achieved using principal component analysis, PCA, an autoencoder, a variational autoencoder, or a deep learning probabilistic framework.

In some embodiments, the method further comprises improving the at least one treatment plan using adaptive radiotherapy techniques.

In some embodiments, the generating of the at least one treatment plan comprises: generating, based on the representation, one or more representative images representing one or more likely potential anatomical states; and generating the at least one treatment plan based on the one or more representative images.

In some embodiments, the generating of the at least one treatment plan further comprises: parameterizing the one or more representative images using a plurality of parameters; and generating the treatment plan based on the parameters.

In some embodiments, the one or more likely potential anatomical states comprises a most likely potential anatomical state, a most likely range of potential anatomical states, and/or an average over the potential anatomical states.

In some embodiments, the most likely potential anatomical state or the most likely range of potential anatomical states is determined using a maximum likelihood estimation (MLE).

In some embodiments, the method further comprises determining a standard deviation from the most likely potential anatomical state, and wherein the most likely range of potential anatomical states is determined based on the standard deviation.

In some embodiments, the most likely range of potential anatomical states is determined such that the most likely range covers a predetermined proportion of the potential anatomical states.

In some embodiments, the predetermined proportion is 99%, 95%, 90%, 85%, 80%, 75% or 70%.

In some embodiments, the one or more representative images are generated such that they are predicted to cover a predetermined proportion of the potential anatomical states.

In some embodiments, the predetermined proportion is 99%, 95%, 90%, 85%, 80%, 75% or 70%.

In some embodiments, the generating of the at least one treatment plan comprises: obtaining, based on the representation, a plurality of reference images, wherein each reference image represents one of the plurality of potential anatomical states; and for each reference image, generating a treatment plan based on the reference image; and wherein the method further comprises training a machine learning model to generate a further treatment plan based on a further image of the patient, the training comprising training the model based on the plurality of reference images and their corresponding treatment plans.

In some embodiments, the method further comprises obtaining a further image of the patient and inputting the further image into the trained model to output the further treatment plan.

In some embodiments, the machine learning model is a patient-specific regression model.

In some embodiments, the reference images and their corresponding treatment plans are specified in a reduced dimensional space.

In some embodiments, the generating of the treatment plan based on the reference image comprises: parameterizing the reference image using a plurality of parameters; and generating the treatment plan based on the parameters.

In some embodiments, the method is used in adaptive radiotherapy.

According to a second aspect of the present disclosure, there is provided a data processing apparatus comprising a memory storing computer-executable instructions, and a processor configured to execute the instructions to carry out a method in accordance with any of the embodiments described above.

According to a third aspect of the present disclosure, there is provided a computer program comprising instructions which, when the program is executed by a computer, cause the computer to carry out a method in accordance with any of the embodiments described above. The computer program may be a computer program product comprising a computer readable medium, the computer readable medium having computer readable code embodied therein, the computer readable code being configured such that, on execution by a suitable computer or processor, the computer or processor is caused to perform a method in accordance with any of the embodiments described above.

According to a fourth aspect of the present disclosure, there is provide a computer-readable medium comprising instructions which, when executed by a computer, cause the computer to carry out a method in accordance with any of the embodiments described above.

Other features of the disclosure are described below.

The invention may be implemented in digital electronic circuitry, or in computer hardware, firmware, software, or in combinations thereof. The invention may be implemented as a computer program or a computer program product, i.e. a computer program tangibly embodied in a non-transitory information carrier, e.g. in a machine-readable storage device or in a propagated signal, for execution by, or to control the operation of, one or more hardware modules.

A computer program may be in the form of a stand-alone program, a computer program portion, or more than one computer program, and may be written in any form of programming language, including compiled or interpreted languages, and it may be deployed in any form, including as a stand-alone program or as a module, component, subroutine, or other unit suitable for use in a data processing environment.

The invention is described in terms of particular embodiments. Other embodiments are within the scope of the following claims. For example, the steps of the invention may be performed in a different order and still achieve desirable results.

Elements of the invention have been described using the terms “processor” etc. The skilled person will appreciate that such functional terms and their equivalents may refer to parts of the system that are spatially separate but combine to serve the function defined. Equally, the same physical parts of the system may provide two or more of the functions defined. For example, separately defined means may be implemented using the same memory and/or processor as appropriate.

### BRIEF DESCRIPTION OF THE DRAWINGS

Embodiments of the invention will now be further described by way of example only and with reference to the accompanying drawings, wherein like reference numerals refer to like parts, and wherein:

FIG. 1 is a flow chart illustrating process steps in a method for generating a radiation treatment plan for a patient for at least one future radiation treatment session.

FIG. 2 is a flow chart illustrating process steps in a method for generating a radiation treatment plan for a patient for at least one future radiation treatment session.

FIG. 3 is a flow chart illustrating process steps in a method for generating a radiation treatment plan for a patient for at least one future radiation treatment session.

FIG. 4 is a flow chart illustrating process steps in a method for generating a radiation treatment plan for a patient for at least one future radiation treatment session.

FIG. 5 is a radiotherapy system, suitable for generating a radiation treatment plan for a patient for at least one future radiation treatment session.

FIG. 6 is a radiotherapy device or apparatus, suitable for implementing a radiation treatment plan generated according to embodiments.

FIG. 7 is a schematic showing examples of anatomical variations which may occur.

FIG. 8 is a schematic of an example treatment plan which may be generated according to embodiments.

### DETAILED DESCRIPTION

Radiation treatment plans are typically based on a 3D reference image, e.g. a CT, CBCT or MRI scan. The reference image used to generate the plan is a static representation of the patient's anatomy, representing a single snapshot out of all the possible anatomical states. Organs are deformable soft tissue structures that can be influenced by a number of processes such as breathing, peristalsis, digestion, and muscle contraction. This results in session-to-session anatomical variations that are often referred to as ‘interfractional motion’, and moment-to-moment variations often referred to as ‘intrafractional motion’. These may be factored into the treatment plan in the form of “margins” which attempt to encapsulate assumptions about the anatomy by creating a region around targets to account for changes.

The use of margins increases the likelihood that the target will receive the intended dose, but the margins may either be too tight, in which case the target may be missed, or too generous, which may unnecessarily increase the dose to healthy tissues, i.e. OARs.

A relatively new technology that is being used in some clinics is online adaptive radiotherapy. A new reference image, such as a CBCT, CT or MRI scan, is acquired each day and used to modify an original treatment plan to better conform the dose distribution to the current patient anatomy. This has the potential to reduce the treatment margins and ensure coverage by those margins, but widespread clinical use is still limited since current implementations add significant time to the session and require physician presence to approve contours and to ensure that the newly adapted plans are clinically acceptable.

Embodiments of the present invention provide methods for generating more robust and representative treatment plans which can reduce margins without necessarily requiring online adaptive radiotherapy and can improve the adaptive radiotherapy process by improving speed, safety and reduce the need for physician presence.

The proposed approach obtains one or more images of a patient (e.g. a single planning CT, a 4D CT, historical images acquired during previous radiotherapy treatments), and then, based on the image(s), generates a representation (e.g. a probability distribution function, reference images, deformation vector fields) of a plurality of anatomical states of the patient which may occur during the future radiation treatment sessions. One or more treatment plans can then be generated based on this representation. For example, the treatment plan may be designed to cover a range of likely anatomical states of the patient. The proposed approach generates robust treatment plans without needing to rely on online radiotherapy.

The plurality of (potential) anatomical states of the patient which may occur during the future radiation treatment sessions refers to the various configurations and positions of anatomical structures of the patient that could possibly arise due to dynamic physiological processes. For example, these possible states may encompass potential anatomical changes resulting from movements caused by breathing, peristalsis, digestion, muscle contractions, and other moment-to-moment variations. The states may also account for continuous and transient alterations in anatomy that occur in response to internal and external stimuli.

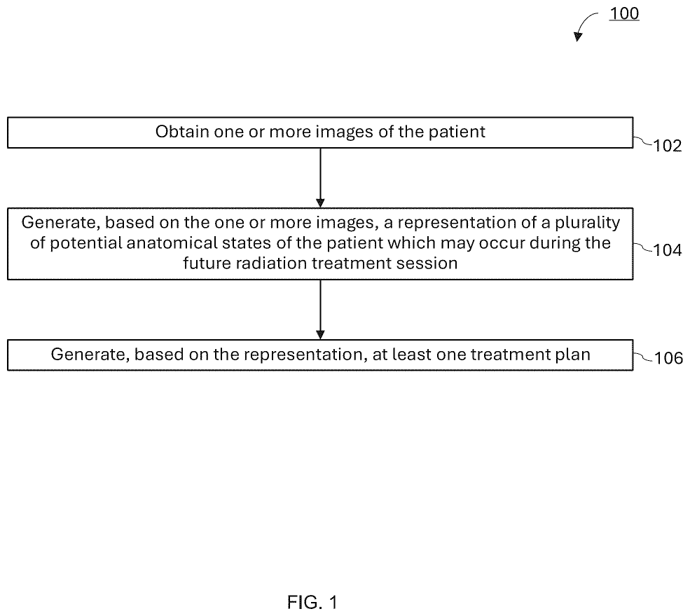

Various aspects and details of these principal concepts will be described below with reference to FIGS. 1 to 8.

FIG. 1 is a flow chart illustrating process steps in a computer implemented method 100 for generating a radiation treatment plan for a patient for at least one future radiation treatment session.

Method 100 comprises, at step 102, obtaining one or more images of the patient. The image(s) may be obtained via measurement of the patient, or via simulation. The image(s) may comprise one or more CT scans of the patient (e.g. a single planning CT scan), one or more 4D CT scans of the patient (e.g. a 4D CT scan may comprise around ten CT scans representing different breathing phases), one or more MRI scans of the patient, and/or one or more 4D MRI scans of the patient. The image(s) may be obtained right before delivering treatment to the patient. Alternatively, the image(s) may be one or more historical images of the patient. For example, the image(s) may have been obtained during a previous assessment or treatment (e.g. during the course of a previous radiotherapy treatment or session).

Method 100 comprises, at step 104, generating, based on the one or more images, a representation of a plurality of potential anatomical states of the patient which may occur during the future radiation treatment session. In one example, step 104 may be performed by an algorithm which accepts M images (i.e. the obtained one or more images) as its input and generates N images Iₙ as output, where Iₙ represent realistic variations of the patient anatomy that could potentially occur in N future treatment fractions. In this example, the representation is in the form of the N images. The output of the algorithm comprises the N images and may optionally be supplemented to include the M input images too.

In some examples, all relevant calculations for generating the treatment plan can be performed based purely on the output data set Iₙ, without requiring construction of a probability density function (PDF). In some examples, the dimensionality of the images in the output data set Iₙ is reduced, for example, using PCA analysis or autoencoder/variational autoencoder to map into a latent space representation. The output image set can then be represented in the reduced dimensional space, or an explicit pdf can be built as a function of the reduced dimensional space.

In some examples, the algorithm used to perform step 104 may output encoded versions of the N images rather than full images. In other examples, the algorithm outputs deformation vector fields (DVFs) or encoded versions of DVFs. For example, each output of the algorithm could represent a DVF that deforms a planning CT to a future treatment fraction image. Reducing the dimensionality of images and/or using encoded versions of images and/or DVFs is advantageous as it can be more computationally efficient and result in reduced processing times.

In some examples, rather than generate N output samples using the algorithm, a PDF or another representation of the probabilistic distribution of outputs may be generated directly. This may be advantageous because the generated PDF may then be optimally sampled such that there is a good balance between not sampling too much (which would be more computationally expensive) while maintaining good coverage of the most likely variations. For example, variational autoencoders (VAEs) can be used to learn a distribution over latent variables representing the images. Other examples include Gaussian processes, Bayesian neural networks (BNNs) or neural fields.

The algorithm used for step 104 may work by generating a plurality of translations and rotations of the one or more images to create the different anatomical states. Additionally, DVFs can be modelled using b-splines and/or optical flow algorithms and/or biomechanical models. The parameters of the models used to model the DVFs can be varied to generate many DVFs. The DVFs may then be used to deform the one or more input images into output images as described above. Using biomechanical models may be advantageous as they can result in more realistic deformations. Alternatively or additionally, statistical population models based on PCA of anatomical changes over a course of treatment can be used.

The algorithm network used for step 104 may be trained on data from multiple patients. The data from each patient may comprise a plurality of images of the patient, acquired at different times. Such data, acquired at different times for the same patient, may be referred to as longitudinal data. For example, longitudinal data may be acquired on different days, or at different times within the same day (e.g. during multiple treatment sessions on one day, or multiple scan sessions on one day), or may even comprise a 4D image which comprises multiple 3D images obtained at different points in the respiratory phase. Each patient's anatomy will vary from day to day, and thus the longitudinal data provides a good training dataset for the algorithm. For example, a recurrent neural network approach may be used, which are particularly suited to handle sequential data and can capture temporal dependencies. Long short-term memory (LSTM) and gated recurrent unit (GRU) are variants that may be beneficial. Variational autoencoders (VAEs) could also be used for this purpose, potentially including temporal dynamics using a temporal VAE or recurrent VAE. Other approaches that could be used are generative adversarial networks (GANs), trained to create realistic variations of the patient's anatomy, while the discriminator ensures that the generated images are realistic. Sequence-to-sequence models (Seq2Seq) with attention mechanisms can be used. A Transformer model can be used to predict future images, leveraging its attention mechanism to capture long-range dependencies and variations; vision transformers (ViTs) or Temporal Transformers are specifically designed for image sequences. Physics-informed neural networks (PINNs) can be used in combination with biomechanical models to generate realistic variations in patient anatomy.

As discussed above, the representation of the plurality of potential anatomical states of the patient may comprise one or more of the following:
a plurality of reference images, wherein each reference image represents one of the plurality of potential anatomical states;
a plurality of encoded reference images, wherein each encoded reference image represents one of the plurality of potential anatomical states;
a plurality of deformation vector fields, DVFs, wherein each DVF deforms the one or more images of the patient to a reference image, wherein each reference image represents one of the plurality of potential anatomical states;
a plurality of encoded DVFs, wherein each encoded DVF deforms the one or more images of the patient to a reference image, wherein each reference image represents one of the plurality of potential anatomical states;
a probabilistic distribution of a plurality of reference images, wherein each reference image represents one of the plurality of potential anatomical states, e.g. a probability density function (PDF) representing a likelihood of the patient being in any one of the potential anatomical states.

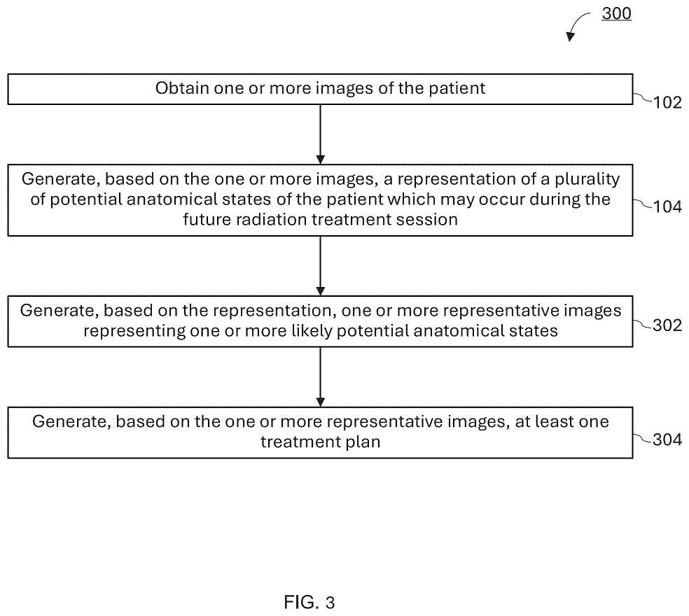

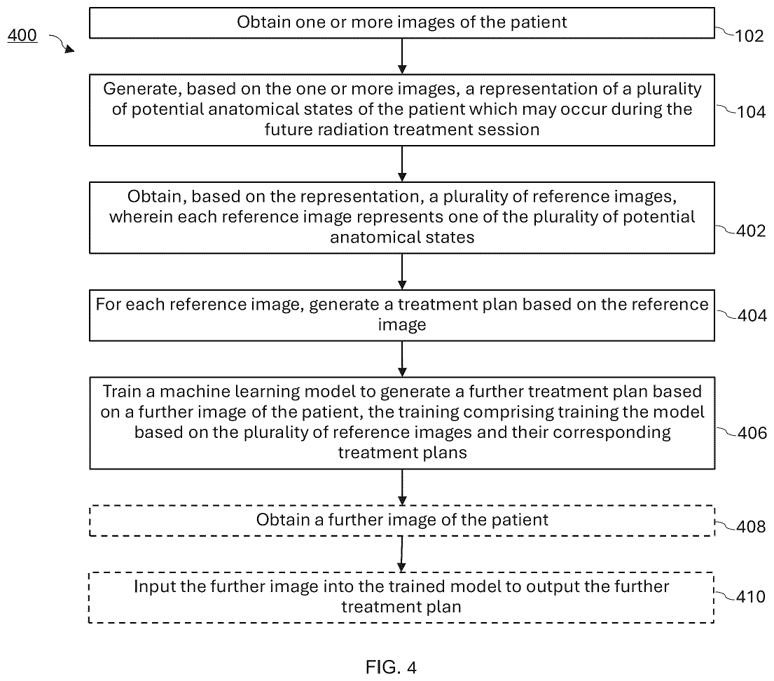

Method 100 comprises, at step 106, generating, based on the representation, at least one treatment plan. This may be done using different approaches, e.g. one approach is described in relation to FIG. 3 (a “most likely” state approach), another approach is described in relation to FIG. 4 (a machine learning model approach).

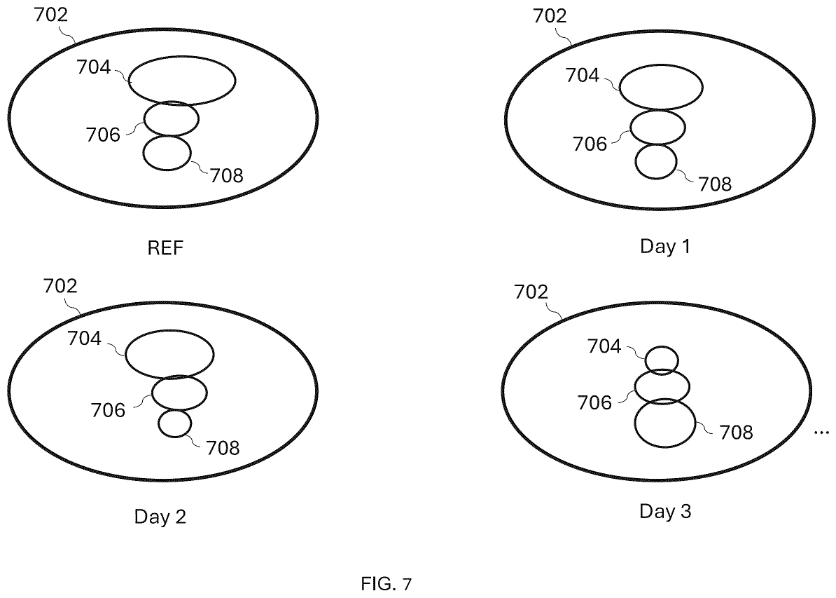

FIG. 7 is a schematic showing different anatomical states to demonstrate how anatomical states of a patient may vary over time. “REF” is a reference day and then Day 1, Day 2 and Day 3 are three different treatment fractions, with prostate 706, rectum 708, bladder 704 and body 702 contours changing over time. For example, in the REF contours, the bladder changes size and shape from the reference day through to day 4. The bladder can change drastically depending on the volume of urine in the bladder at the time of the imaging/treatment. The prostate does not change size drastically, but may shift and rotate alongside small volume changes. The rectum changes size and shape primarily due to rectal filling and gas bubble presence. As such, the contours of different body parts vary over time. As an example, the representation of the plurality of potential anatomical states generated in step 104 may comprise such states shown in FIG. 7. While FIG. 7 refers to the prostate, rectum and bladder, embodiments of the present invention are applicable to any organs or anatomical structures which may change over time.

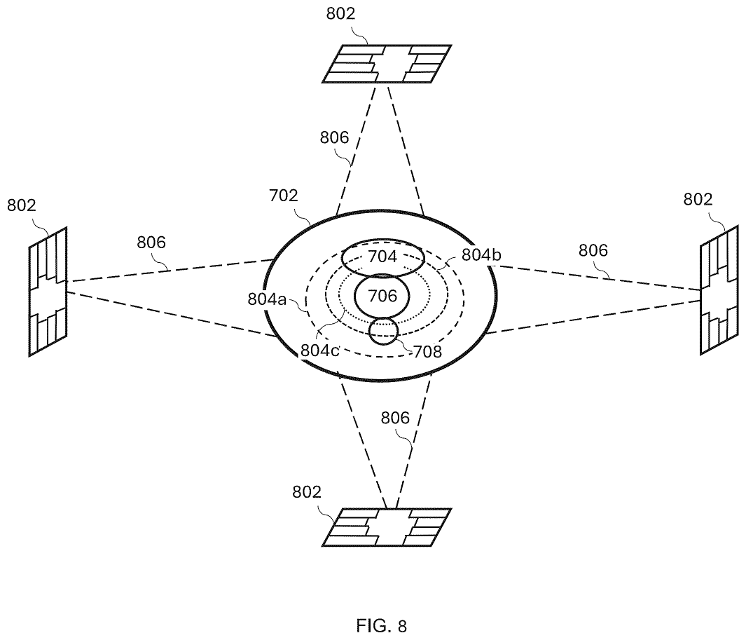

FIG. 8 shows a simplified representation of what the at least one treatment plan may look like. The contours of the body 702, prostate 706, rectum 708 and bladder 704 can be seen, similarly to FIG. 7. FIG. 8 shows four multi-leaf collimator (MLC) apertures which shape the radiation beams 806 of the linac to match the outline of the tumour from different angles. By moving the individual leaves of the MLC, the MLC blocks certain parts of the beam, allowing only specific sections to pass through. MLCs allow for precise delivery of radiation to the tumour, sparing nearby healthy tissue. This precision is important in techniques like Intensity-Modulated Radiation Therapy (IMRT) and Volumetric Modulated Arc Therapy (VMAT), where the beam intensity can be modulated to match the tumour's 3D shape. Isodose lines 804abc are contour lines representing areas within the body 702 receiving the same dose of radiation. MLCs help tailor the radiation beam so that the isodose lines tightly conform to the shape of the tumour, all the beams 806 combine to generate the dose distribution. In this simplified representation, only four MLC apertures/beams are shown. In practice there would usually be more than four beams, e.g. nine. Or, if VMAT is used, the gantry of the linac moves around the patient, delivering radiation in a continuous arc. The MLC apertures may change over time as the treatment is delivered, to sculpt the dose distribution, which is shown as an isodose distribution (isodose lines 804abc). As such, the generated at least one treatment plan may comprise one or more of: an indication of how many radiation beams 806 are needed, how the MLC leaves should be positioned for the treatment, the isodose contours 804abc, and a likely anatomical state 702, 704, 706, 708 of the patient.

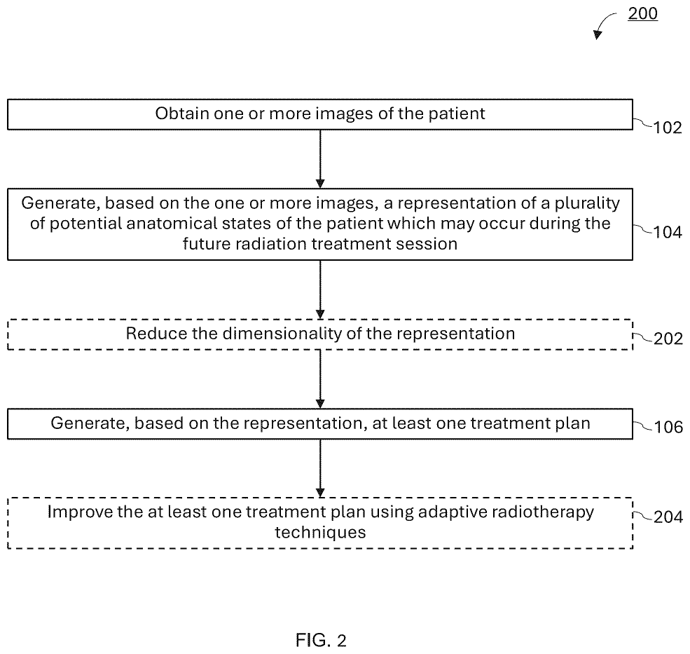

FIG. 2 is a flow chart illustrating process steps in a computer implemented method 200 for generating a radiation treatment plan for a patient for at least one future radiation treatment session. Method 200 is identical to method 100 described above, with the addition of one or more of optional steps 202 and 204. As such, method 200 comprises steps 102, 104, optionally step 202, step 106, and optionally step 204.

Between step 104 and 106, method 200 may comprise optional step 202. Optional step 202 comprises reducing the dimensionality of the representation, as mentioned above, reducing the dimensionality can have positive effects on computational efficiencies and computation times. This may be performed prior to generating the at least one treatment plan. Reducing the dimensionality of the representation may be achieved using principal component analysis, PCA, an autoencoder, a variational autoencoder, or a deep learning probabilistic framework. The article “A probabilistic deep learning model of inter-fraction anatomical variations in radiotherapy” by Pastor-Serrano et al. (DOI: 10.1088/1361-6560/acc71d) discusses various approaches which could be used for this purpose. They model deformations using deformation vector fields (DVF), warping a patient's planning CT into possible patient-specific anatomies based on a few parameters capturing groups of correlated movements. Once the dimensionality has been reduced, it is more computationally tractable to express the PDF. This is useful because the dimensionality of the PDF, for example, will be very high-one dimension per image voxel.

After step 106, method 200 may comprise optional step 204. Optional step 204 comprises improving the at least one treatment plan using adaptive radiotherapy techniques. Examples of suitable adaptive radiotherapy techniques include: segment aperture morphing (SAM) with or without beam weight re-optimization, e.g. Ahunbay, Ergun E., et al. “An on-line replanning scheme for interfractional variations a.” Medical physics 35.8 (2008): 3607-3615; an adaptive sequencer such as C. Kontaxis, G. Bol, J. Lagendijk, B. Raaymakers, Towards adaptive IMRT sequencing for the MR-linac, Physics in Medicine & Biology, 60 (2015) 2493; or, the treatment plan may be run again from scratch using any available treatment planning algorithm.

FIG. 3 is a flow chart illustrating process steps in a computer implemented method 300 for generating a radiation treatment plan for a patient for at least one future radiation treatment session. Method 300 contains steps 102 and 104 described above in relation to method 100, and then steps 302 and 304.

Step 302 comprises generating, based on the representation (from step 104, which may be a set of images Iₙ, or some other higher description of the probability of obtaining a particular image/(e.g. a PDF)), one or more representative images representing one or more likely potential anatomical states. Potential anatomical states may account for day-to-day variations such as bladder filling (e.g. bladder 704 changes in size over the daily images schematically illustrated in FIG. 7), rectal filling (e.g. rectum 708 changes in size over the daily images schematically illustrated in FIG. 7), air pockets, digestion, different patient positions, tumour growth which can influence the whole surrounding area, weight gain/loss, peristalsis, etc. These representative images are images which can be used for treatment planning.

For example, step 302 may generate a “most likely” image using maximum likelihood estimation (i.e. the one or more representative images comprise the most likely image), or calculate an average image (i.e. the one or more representative images comprise the average image), or select an image at the centre of a latent space representation (i.e. the one or more representative images comprise this centre image), or use a PDF as a function of a reduced dimensional space to find the most likely image (i.e. the one or more representative images comprise this most likely image). Images that represent extreme scenarios (i.e. rare scenarios, e.g. where anatomical variations are larger than 2 cm) can also be generated to ensure the planning is robust for those images and thus covers a wide range of scenarios (i.e. the one or more representative images may include images representing extreme scenarios).

As such, the one or more likely potential anatomical states may comprise a (single) most likely potential anatomical state, a most likely range of potential anatomical states, and/or an average over the potential anatomical states. The (single) most likely potential anatomical state or the most likely range of potential anatomical states may be determined using a maximum likelihood estimation (MLE). The most likely range of potential anatomical states may be determined based on a standard deviation calculated from the (single) most likely potential anatomical state. The most likely range of potential anatomical states may be determined such that the most likely range covers a predetermined proportion of the potential anatomical states. For example, the predetermined proportion may be 99%, 95%, 90%, 85%, 80%, 75% or 70% of the potential anatomical states (i.e. the most likely range may cover 95% of the potential anatomical states). The one or more representative images may be generated such that they are predicted to cover a predetermined proportion of the potential anatomical states. For example, the predetermined proportion may be 99%, 95%, 90%, 85%, 80%, 75% or 70% of the potential anatomical states (i.e. the representative images may cover 95% of the potential anatomical states).

Step 304 comprises generating at least one treatment plan based on the one or more representative images. For example, if the representative image is a “most likely” single image, then a single treatment plan for that most likely image may be generated. In another example, if the representative images comprise a plurality of images (e.g. a most likely range) then a treatment plan may be generated to cover the plurality of images so that it would be an acceptable plan for every eventuality (or a significant fraction thereof) covered in the plurality of images. Alternatively, multiple treatment plans may be generated in order to cover the plurality of images. As such, step 304 may comprise generating a treatment plan per representative image, or optimising one treatment plan over a plurality of representative images. This creates a “plan of the day” for a multitude of different anatomical states. The treatment plan(s) generated based on the representative image(s) can be generated using standard treatment planning technology, e.g. Autoplan technology.

Step 304 may comprise parameterizing the one or more representative images using a plurality of parameters, and then generating the treatment plan based on the parameters. This is advantageous as it addresses the issue is that there may be many treatment plans generated, potentially one for each of many images used. These plans need to somehow be evaluated and deemed as clinically acceptable by the physician. There may be too many treatment plans to approve in a short period of time. Advantageously, images, contours and plans can be labelled by a small number of parameters. Contour, plan and dose metrics can then be analysed and displayed to a user (e.g. a physician) as a function of these parameters. In this way, physicians can approve all (or at least many) of the possible situations that will occur in any give fraction up front, and thus do not need to be present for each adaptive radiotherapy treatment session. For example, a physician may approve a range of parameter values for a given parameter—e.g. if parameter A is between x and y, it is approved. In a given adaptation session, an image may first be acquired, parametrized in terms of the few parameters describing the degrees of freedom, and then contours and plans would be generated from these parameters. If the image is not able to be parametrized in terms of the parameters, then conventional contouring and planning techniques may be used instead, but this would likely be for a relatively small number of cases.

As such, method 300 can be used to generate a treatment plan which covers the most likely anatomical variations to occur during the treatment. The treatment plans generated can be robust for many different scenarios and can be quickly generated based on as little as a single image of the patient.

FIG. 4 is a flow chart illustrating process steps in a computer implemented method 400 for generating a radiation treatment plan for a patient for at least one future radiation treatment session. Method 400 contains steps 102 and 104 described above in relation to method 100, and then steps 402, 404 and 406, and then optional steps 408 and 410.

Step 402 comprises obtaining, based on the representation (from step 104, which may be a set of images Iₙ, or some other higher description of the probability of obtaining a particular image/(e.g. a PDF)), a plurality of reference images I, wherein each reference image represents one of the plurality of potential anatomical states. Each reference image may be specified in a reduced dimensional space, as described above.

Step 404 comprises, for each reference image Iₙ, generating a treatment plan Tₙ based on the reference image. This generation of the treatment plans can be done automatically. Each treatment plan may be specified in a reduced dimensional space. Step 404 may comprise parameterizing the reference image using a plurality of parameters, and then generating the treatment plan based on the parameters. As discussed above, parameterizing helps address the issue of physicians needing to clinically approve each generated treatment plan.

Step 406 comprises training a machine learning model to generate a further treatment plan based on a further image of the patient, the training comprising training the model based on the plurality of reference images and their corresponding treatment plans. The model is trained to relate images I and treatment plans T. For example, an automatic treatment plan Tₙ can be generated for each image in the set of potential realizations of the images Iₙ. The network can be trained using this paired patient specific data. The plurality of reference images and their corresponding treatment plans may be referred to as a training data set. Using the training data set to train the machine learning model may comprise using the training data set to update values of trainable parameters of the machine learning model. This may comprise updating values of the trainable parameters so as to minimise, for a given image, a loss function based on a difference between the corresponding treatment plan for the image from the training dataset, and the predicted treatment plan output by the machine learning model. The machine learning model may be a patient-specific regression model. Once trained, the machine learning model can then take an input image and output an appropriate treatment plan. The images and treatment plans may be specified in a reduced dimensional space. The machine learning model may be trained offline. This offline training helps to speed up the live treatment planning process as the machine learning model is already trained on existing data, overcoming bottlenecks in existing approaches which are not amenable to real time exploration and optimization.

For the purposes of the present disclosure, the term “machine learning model” encompasses within its scope the following concepts:
machine learning algorithms, comprising processes or instructions through which data may be used in a training process to generate a model artefact for performing a given task, or for representing a real-world process or system; and
the model artefact that is created by such a training process, and which comprises the computational architecture that performs the task.

Optional step 408 comprises obtaining a further image of the patient. This further image may be obtained during a particular adaptive radiotherapy session which needs a treatment plan to be generated based on the up-to-date further image.

Optional step 410 comprises inputting the further image into the trained model to output the further treatment plan (the most appropriate treatment plan for the further image). This allows the rapid determination of a treatment plan for adaptive radiotherapy without the need to re-optimise. Where the model is used to generate a new treatment plan based on a new image, the treatment plan may be further adapted to the image using a more traditional optimization algorithm (e.g. using known adaptive radiotherapy techniques such as techniques including gradient descent and/or simulated annealing).

Method 400 enables rapid generation of a treatment plan based on an image of the patient. Within a particular adaptive radiotherapy session, a patient may be imaged, the image may be input into the trained machine learning model (the model previously being trained on the patient's previous data so the model is patient-specific), and the model quickly outputs a treatment plan for that patient for that particular adaptive radiotherapy session.

Any of the methods 100, 200, 300, 400 described above may be used in combination with adaptive radiotherapy. For example, known adaptive radiotherapy techniques (e.g. warm start optimisation) may be used to improve the generated treatment plans even further. When the methods disclosed herein are used in combination with adaptive radiotherapy, the speed of adaptation is increased and the need for physician presence is reduced, which are current bottlenecks in widespread adoption of the technology.

Example methods according to the present disclosure allow fast and robust treatment plan generation for radiotherapy treatment. The methods described above offer speed advantages associated with use of machine learning and produce patient-specific models which consequently can be applied to quickly generate a treatment plan for a specific patient. This combination of speed and quality can support both the planning and delivery of radiotherapy treatment, for example in the form of online Adaptive Radiotherapy (ART).

The robust treatment plans and speed afforded by methods of the present disclosure can support the provision of online Adaptive Radiotherapy (ART), in which CBCT is used to capture patient imaging at the start of each visit of the treatment fraction. This up-to-date imaging data can enable clinicians to track changes in patient anatomy, including for example, tumour shrinkage over the course of the radiotherapy treatment, allowing for online target localisation and plan adaptation without the constraints of diagnostic CT imaging. For example, this up-to-date imaging data may be input (see step 410) into a trained patient-specific machine learning model according to the present disclosure and used to generate an up-to-date robust treatment plan for the patient. The treatment plan generation offered by methods according to the present disclosure may result in many additional medical treatment benefits (including improved accuracy of radiotherapy treatment, reduced exposure to unintended radiation, reduced treatment duration, etc.). The methods presented herein may be applicable to a variety of medical treatment and diagnostic settings or radiotherapy treatment equipment and devices.

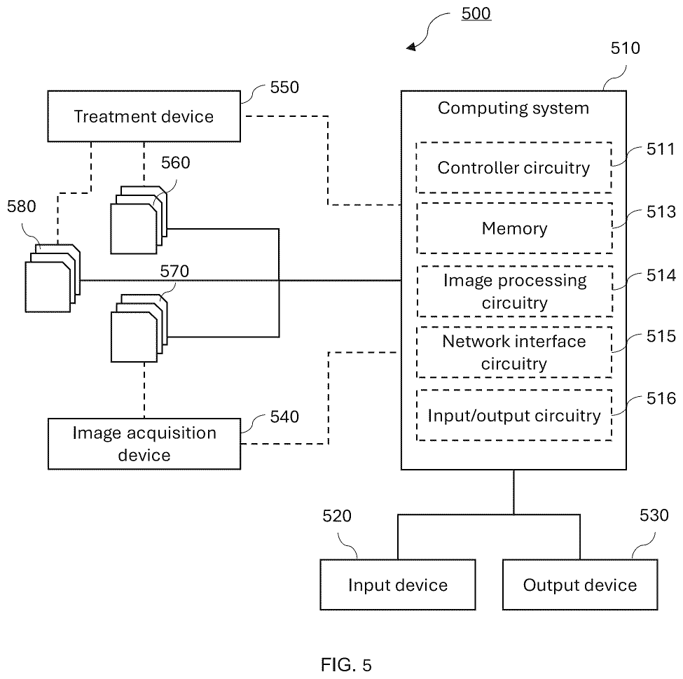

FIG. 5 is a block diagram of an implementation of a radiotherapy system 500, suitable for executing methods according to embodiments (e.g, methods 100, 200, 300, 400). The example radiotherapy system 500 comprises a computing system 510 within which a set of instructions, for causing the computing system 510 to perform the method (or steps thereof) discussed herein, may be executed.

The computing system 510 may implement a planning system arranged to generate radiation treatment plans, e.g. as described in relation to methods 100, 200, 300, 400. The computing system 510 may also be referred to as a computer. In particular, the methods described herein may be implemented by a processor or controller circuitry 511 of the computing system 510.

The computing system 510 shall be taken to include any number or collection of machines, e.g., computing device(s), that individually or jointly execute a set (or multiple sets) of instructions to perform any one or more of the methods discussed herein. That is, hardware and/or software may be provided in a single computing device, or distributed across a plurality of computing devices in the computing system. In some implementations, one or more elements of the computing system may be connected (e.g., networked) to other machines, for example in a Local Area Network (LAN), an intranet, an extranet, or the Internet. One or more elements of the computing system may operate in the capacity of a server or a client machine in a client-server network environment, or as a peer machine in a peer-to-peer (or distributed) network environment. One or more elements of the computing system may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine.

The computing system 510 includes controller circuitry 511 and a memory 513 (e.g., read-only memory (ROM), flash memory, dynamic random access memory (DRAM) such as synchronous DRAM (SDRAM) or Rambus DRAM (RDRAM), etc.). The memory 513 may comprise a static memory (e.g., flash memory, static random access memory (SRAM), etc.), and/or a secondary memory (e.g., a data storage device), which communicate with each other via a bus (not shown). Memory 513 may be used to store or buffer image data until required for processing.

Controller circuitry 511 represents one or more general-purpose processors such as a microprocessor, central processing unit, accelerated processing units, or the like. More particularly, the controller circuitry 511 may comprise a complex instruction set computing (CISC) microprocessor, reduced instruction set computing (RISC) microprocessor, very long instruction word (VLIW) microprocessor, processor implementing other instruction sets, or processors implementing a combination of instruction sets. Controller circuitry 511 may also include one or more special-purpose processing devices such as an application specific integrated circuit (ASIC), a field programmable gate array (FPGA), a digital signal processor (DSP), network processor, or the like. One or more processors of the controller circuitry may have a multicore design. Controller circuitry 511 is configured to execute the processing logic for performing the operations and steps discussed herein.

The computing system 510 may further include a network interface circuitry 515. The computing system 510 may be communicatively coupled to an input device 520 and/or an output device 530, via input/output circuitry 516. In some implementations, the input device 520 and/or the output device 530 may be elements of the computing system 510. The input device 520 may include an alphanumeric input device (e.g., a keyboard or touchscreen), a cursor control device (e.g., a mouse or touchscreen), an audio device such as a microphone, and/or a haptic input device. The output device 530 may include an audio device such as a speaker, a video display unit (e.g., a liquid crystal display (LCD) or a cathode ray tube (CRT)), and/or a haptic output device. In some implementations, the input device 520 and the output device 530 may be provided as a single device, or as separate devices.

In some implementations, the computing system 510 may comprise image processing circuitry 514. Image processing circuitry 514 may be configured to process image data 570 (e.g., images, imaging data, projections, projection data), such as medical images obtained from one or more imaging data sources, a treatment device 550 and/or an image acquisition device 540. Image processing circuitry 514 may be configured to process, or pre-process, image data 570. For example, image processing circuitry 514 may convert received image data into a particular format, size, resolution or the like. Image processing circuitry 514 may be configured to perform image reconstruction. In some implementations, image processing circuitry 514 may be combined with controller circuitry 511.

In some implementations, the radiotherapy system 500 may further comprise an image acquisition device 540 and/or a treatment device 550. The image acquisition device 540 and the treatment device 550 may be provided as a single device. In some implementations, treatment device 550 is configured to perform imaging, for example in addition to providing treatment and/or during treatment.

Image acquisition device 540 may be configured to perform positron emission tomography (PET), computed tomography (CT), magnetic resonance imaging (MRI), single positron emission computed tomography (SPECT), X-ray, and the like.

Image acquisition device 540 may be configured to output image data 570, which may be accessed by computing system 510. Treatment device 550 may be configured to output treatment data 560, which may be accessed by computing system 510.

Computing system 510 may be configured to access or obtain treatment data 560, planning data 580 and/or image data 570. Treatment data 560 may be obtained from an internal data source (e.g., from memory 513) or from an external data source, such as treatment device 550 or an external database. Planning data 580 may be obtained from memory 513 and/or from an external source, such as a planning database. Planning data 580 may comprise information obtained from one or more of the image acquisition device 540 and the treatment device 550.

The various methods described above (e.g, methods 100, 200, 300, 400) may be implemented by a computer program. The computer program may include computer code (e.g., instructions) arranged to instruct a computer to perform the functions of one or more of the various methods described above. For example, the steps of the methods described in relation to any of FIGS. 1 to 4 may be performed by the computer code. The steps of the methods described above may be performed in any suitable order. The computer program and/or the code for performing such methods may be provided to an apparatus, such as a computer, on one or more computer readable media or, more generally, a computer program product. The computer readable media may be transitory or non-transitory. The one or more computer readable media could be, for example, an electronic, magnetic, optical, electromagnetic, infrared, or semiconductor system, or a propagation medium for data transmission, for example for downloading the code over the Internet. Alternatively, the one or more computer readable media could take the form of one or more physical computer readable media such as semiconductor or solid-state memory, magnetic tape, a removable computer diskette, a random access memory (RAM), a read-only memory (ROM), a rigid magnetic disc, and an optical disk, such as a CD-ROM, CD-R/W or DVD. The instructions may also reside, completely or at least partially, within the memory 513 and/or within the controller circuitry 511 during execution thereof by the computing system 510, the memory 513 and the controller circuitry 511 also constituting computer-readable storage media.

In an implementation, the modules, components and other features described herein may be implemented as discrete components or integrated in the functionality of hardware components such as ASICS, FPGAs, DSPs or similar devices.

A “hardware component” is a tangible (e.g. non-transitory) physical component (e.g. a set of one or more processors) capable of performing certain operations (such as the steps outlined in methods 100, 200, 300, 400) and may be configured or arranged in a certain physical manner. A hardware component may include dedicated circuitry or logic that is permanently configured to perform certain operations. A hardware component may comprise a special-purpose processor, such as an FPGA or an ASIC. A hardware component may also include programmable logic or circuitry that is temporarily configured by software to perform certain operations.

In addition, the modules and components may be implemented as firmware or functional circuitry within hardware devices. Further, the modules and components may be implemented in any combination of hardware devices and software components, or only in software (e.g. code stored or otherwise embodied in a machine-readable medium or in a transmission medium).

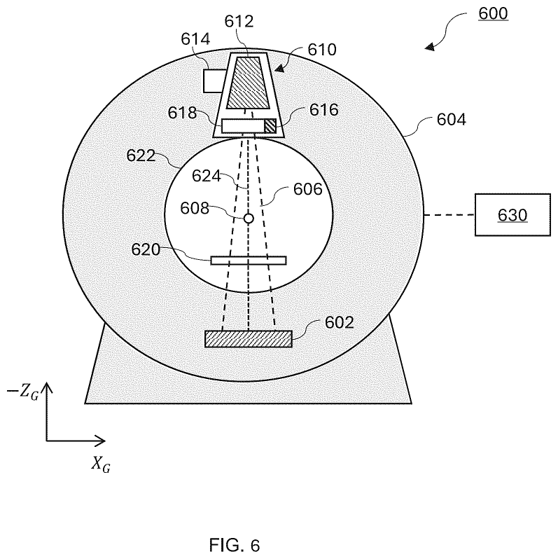

FIG. 6 depicts a radiotherapy apparatus, suitable for implementing radiation treatment plans generated by embodiments. The cross-section through radiotherapy apparatus 600 includes a radiation head 610 and a beam receiving apparatus 602, both of which are attached to a gantry 604. The radiation head 610 includes a radiation source 612, which emits a beam of radiation 606. The radiation head 610 also includes a beam shaping apparatus 618, which controls the size and shape of the radiation field associated with the beam.

The beam receiving apparatus 602 is configured to receive radiation emitted from the radiation head 610, for the purpose of absorbing and/or measuring the beam of radiation. In the view shown, the radiation head 610 and the beam receiving apparatus 602 are positioned diametrically opposed to one another.

The gantry 604 is rotatable and supports the radiation head 610 and the beam receiving apparatus 602 such that they are rotatable around an axis of rotation 608, which may coincide with the patient longitudinal axis. The gantry provides rotation of the radiation head 610 and the beam receiving apparatus 602 in a plane perpendicular to the patient longitudinal axis (e.g., a sagittal plane). Three gantry directions X_{G}, Y_{G}, Z_{G} may be defined, where the Y_{G} direction is perpendicular with gantry axis of rotation. The Z_{G} direction extends from a point on the gantry corresponding to the radiation head, towards the axis of rotation of the gantry. Therefore, from the patient frame of reference, the Z_{G} direction rotates around as the gantry rotates.

Radiotherapy apparatus 600 also includes a support surface 620 on which a subject (or patient) is supported during radiotherapy treatment. The radiation head 610 is configured to rotate around the axis of rotation 608 such that the radiation head 610 directs radiation towards the subject from various angles around the subject in order to spread out the radiation dose received by healthy tissue to a larger region of healthy tissue while building up a prescribed dose of radiation at a target region.

The radiotherapy apparatus 600 is configured to deliver a radiation beam towards a radiation isocentre, which is substantially located on the axis of rotation 608 at the centre of the gantry 604 regardless of the angle at which the radiation head 610 is placed.

The rotatable gantry 604 and radiation head 610 are dimensioned so as to allow a central bore 622 to exist. The central bore 622 provides an opening, sufficient to allow a subject to be positioned therethrough without the possibility of being incidentally contacted by the radiation head 610 or other mechanical components as the gantry rotates the radiation head 610 about the subject.

The radiation head 610 emits the radiation beam 606 along a beam axis 624 (or radiation axis or beam path), where the beam axis 624 is used to define the direction in which the radiation is emitted by the radiation head. The radiation beam 606 is incident on the beam receiving apparatus 602, which may include at least one of a beam stopper and a radiation detector. The beam receiving apparatus 602 is attached to the gantry 604 on a diametrically opposite side to the radiation head 610 to attenuate and/or detect a beam of radiation after the beam has passed through the subject.

The radiation beam axis 624 may be defined as, for example, a centre of the radiation beam 606 or a point of maximum intensity.

The beam shaping apparatus 618 delimits the spread of the radiation beam 606. The beam shaping apparatus 618 is configured to adjust the shape and/or size of a field of radiation produced by the radiation source. The beam shaping apparatus 618 does this by defining an aperture (also referred to as a window or an opening) of variable shape to collimate the radiation beam 606 to a chosen cross-sectional shape. In this example, the beam shaping apparatus 618 may be provided by a combination of a diaphragm and an MLC. Beam shaping apparatus 618 may also be referred to as a beam modifier.

The radiotherapy apparatus 600 may be configured to deliver both coplanar and non-coplanar (also referred to as tilted) modes of radiotherapy treatment. In coplanar treatment, radiation is emitted in a plane which is perpendicular to the axis of rotation of the radiation head 610. In non-coplanar treatment, radiation is emitted at an angle which is not perpendicular to the axis of rotation. In order to deliver coplanar and non-coplanar treatment, the radiation head 610 may move between at least two positions, one in which the radiation is emitted in a plane which is perpendicular to the axis of rotation (coplanar configuration) and one in which radiation is emitted in a plane which is not perpendicular to the axis of rotation (non-coplanar configuration).

In the coplanar configuration, the radiation head is positioned to rotate about a rotation axis and in a first plane. In the non-coplanar configuration, the radiation head is tilted with respect to the first plane such that a field of radiation produced by the radiation head is directed at an oblique angle relative to the first plane and the rotation axis. In the non-coplanar configuration, the radiation head is positioned to rotate in a respective second plane parallel to and displaced from the first plane. The radiation beam is emitted at an oblique angle with respect to the second plane, and therefore as the radiation head rotates the beam sweeps out a cone shape.

The beam receiving apparatus 602 remains in the same place relative to the rotatable gantry when the radiotherapy apparatus is in both the coplanar and non-coplanar modes. Therefore, the beam receiving apparatus 602 is configured to rotate about the rotation axis in the same plane in both coplanar and non-coplanar modes. This may be the same plane as the plane in which the radiation head rotates.

The beam shaping apparatus 610 is configured to reduce the spread of the field of radiation in the non-coplanar configuration in comparison to the coplanar configuration.

The radiotherapy apparatus 600 includes a controller 630, which is programmed to control the radiation source 612, beam receiving apparatus 602 and the gantry 604. Controller 630 may perform functions or operations such as treatment planning, treatment execution, image acquisition, image processing, motion tracking, motion management, and/or other tasks involved in a radiotherapy process.

Controller 630 is programmed to control features of apparatus 600 according to a radiotherapy treatment plan for irradiating a target region, also referred to as a target tissue, of a patient. The treatment plan includes information about a particular dose to be applied to a target tissue, as well as other parameters such as beam angles, dose-histogram-volume information, the number of radiation beams to be used during therapy, the dose per beam, and the like. Controller 630 is programmed to control various components of apparatus 600, such as gantry 604, radiation head 610, beam receiving apparatus 602, and support surface 620, according to the treatment plan. The treatment plan may be generated using contours determined according to embodiments.

Hardware components of controller 630 may include one or more computers (e.g., general purpose computers, workstations, servers, terminals, portable/mobile devices, etc.); processors (e.g., central processing units (CPUs), graphics processing units (GPUs), microprocessors, digital signal processors (DSPs), field programmable gate arrays (FPGAs), special-purpose or specially-designed processors, etc.); memory/storage devices such as a memory (e.g., read-only memories (ROMs), random access memories (RAMs), flash memories, hard drives, optical disks, solid-state drives (SSDs), etc.); input devices (e.g., keyboards, mice, touch screens, mics, buttons, knobs, trackballs, levers, handles, joysticks, etc.); output devices (e.g., displays, printers, speakers, vibration devices, etc.); circuitries; printed circuit boards (PCBs); or other suitable hardware. Software components of controller 630 may include operation device software, application software, etc.

The radiation head 610 may be connected to a head actuator 614, which is configured to actuate the radiation head 610, for example between a coplanar configuration and one or more non-coplanar configurations. This may involve translation and rotation of the radiation head 610 relative to the gantry. In some implementations, the head actuator may include a curved rail along which the radiation head 610 may be moved to adjust the position and angle of the radiation head 610. The controller 630 may control the configuration of the radiation head 630 via the head actuator 614.

The beam shaping apparatus 618 includes a shaping actuator 616. The shaping actuator is configured to control the position of one or more elements in the beam shaping apparatus 618 in order to shape the radiation beam 606. In some implementations, the beam shaping apparatus 618 includes an MLC, and the shaping actuator 616 includes means for actuating leaves of the MLC. The beam shaping apparatus 618 may further comprise a diaphragm, and the shaping actuator 616 may include means for actuating blocks of the diaphragm. The controller 630 may control the beam shaping apparatus 618 via the shaping actuator 616.

A treatment plan may comprise positioning information of beam shaping apparatus 618. The positioning information of beam shaping apparatus 618 may comprise information indicating a configuration of one or more elements of beam shaping apparatus 618, such as leaf configuration of an MLC of beam shaping apparatus 618, a configuration of a diaphragm of beam shaping apparatus 618, a configuration of an opening (e.g., window or aperture) of the MLC, and/or the like.

Unless specifically stated otherwise, as apparent from the following discussion, it is appreciated that throughout the description, discussions utilizing terms such as “receiving”, “determining”, “comparing”, “generating”, “modelling”, “reducing”, “obtaining”, “parameterizing”, “training”, or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

While certain embodiments have been described, these embodiments have been presented by way of example only and are not intended to limit the scope of the inventions. Indeed, the novel methods and apparatuses described herein may be embodied in a variety of other forms; furthermore, various omissions, substitutions and changes in the form of methods and apparatus described herein may be made.

1. A computer-implemented method for generating a radiation treatment plan for a patient for at least one future radiation treatment session, the method comprising:
obtaining one or more images of the patient;
generating, based on the one or more images, a representation of a plurality of potential anatomical states of the patient which may occur during the future radiation treatment session; and
generating, based on the representation, at least one treatment plan.

2. The method of claim 1, wherein the generating of the representation comprises one or more of the following:
generating one or more translations and one or more rotations of the one or more images; and/or
modelling deformation vector fields, DVFs, and optionally wherein the modelling of the DVFs comprises using b-splines and/or optical flow algorithms and/or biomechanical models.

3. The method of claim 1, wherein the generating of the representation uses a network trained on data from multiple patients, the data from each patient comprising a plurality of images of the patient, acquired at different times.

4. The method of claim 3, wherein the network comprises one or more of:
a recurrent neural network, and preferably a long short-term memory, LTSM, recurrent neural network or a gated recurrent unit, GRU, recurrent neural network;
a variational autoencoder, VAE, and preferably a temporal VAE or a recurrent VAE;
a generative adversarial network, GAN;
a sequence-to-sequence model with an attention mechanism;
a transformer model;
a vision transformer model;
a temporal transformer model;
a physics-informed neural network, PINN.

5. The method of claim 1, wherein the representation of the plurality of potential anatomical states of the patient comprises one or more of the following:
a plurality of reference images, wherein each reference image represents one of the plurality of potential anatomical states;
a plurality of encoded reference images, wherein each encoded reference image represents one of the plurality of potential anatomical states;
a plurality of deformation vector fields, DVFs, wherein each DVF deforms the one or more images of the patient to a reference image, wherein each reference image represents one of the plurality of potential anatomical states;
a plurality of encoded DVFs, wherein each encoded DVF deforms the one or more images of the patient to a reference image, wherein each reference image represents one of the plurality of potential anatomical states;
a probabilistic distribution of a plurality of reference images, wherein each reference image represents one of the plurality of potential anatomical states, and preferably wherein the probabilistic distribution comprises a probability density function, PDF, representing a likelihood of the patient being in any one of the potential anatomical states.

6. The method of claim 1, wherein the one or more images of the patient comprise one or more of the following:
one or more images obtained via measurement of the patient;
one or more images obtained via simulation;
one or more CT scans of the patient;
one or more 4D CT scans of the patient;
one or more MRI scans of the patient;
one or more 4D MRI scans of the patient;
one or more historical images of the patient.

7. The method of claim 1, wherein the method further comprises reducing the dimensionality of the representation, prior to generating the at least one treatment plan; and optionally wherein reducing the dimensionality of the representation is achieved using principal component analysis, PCA, an autoencoder, a variational autoencoder, or a deep learning probabilistic framework.

8. The method of claim 1, wherein the method further comprises improving the at least one treatment plan using adaptive radiotherapy techniques.

9. The method of claim 1, wherein the generating of the at least one treatment plan comprises:
generating, based on the representation, one or more representative images representing one or more likely potential anatomical states; and
generating the at least one treatment plan based on the one or more representative images.

10. The method of claim 9, wherein the generating of the at least one treatment plan further comprises:
parameterizing the one or more representative images using a plurality of parameters; and
generating the treatment plan based on the parameters.

11. The method of claim 9, wherein the one or more likely potential anatomical states comprises a most likely potential anatomical state, a most likely range of potential anatomical states, and/or an average over the potential anatomical states.

12. The method of claim 9, wherein the one or more representative images are generated such that they are predicted to cover a predetermined proportion of the potential anatomical states; and optionally wherein the predetermined proportion is 99%, 95%, 90%, 85%, 80%, 75% or 70%.

13. The method of claim 1, wherein, the generating of the at least one treatment plan comprises:
obtaining, based on the representation, a plurality of reference images, wherein each reference image represents one of the plurality of potential anatomical states; and
for each reference image, generating a treatment plan based on the reference image; and wherein the method further comprises training a machine learning model to generate a further treatment plan based on a further image of the patient, the training comprising training the model based on the plurality of reference images and their corresponding treatment plans.

14. The method of claim 13, wherein the method further comprises obtaining a further image of the patient and inputting the further image into the trained model to output the further treatment plan.

15. The method of claim 13, wherein the machine learning model is a patient-specific regression model.

16. The method of claim 13, wherein the reference images and their corresponding treatment plans are specified in a reduced dimensional space.

17. The method of claim 13, wherein, the generating of the treatment plan based on the reference image comprises:
parameterizing the reference image using a plurality of parameters; and
generating the treatment plan based on the parameters.

18. The method of claim 1, for use in adaptive radiotherapy.

19. A data processing apparatus for generating a radiation treatment plan for a patient for at least one future radiation treatment session, the data processing apparatus comprising a memory storing computer-executable instructions, and a processor configured to execute the instructions to:
obtain one or more images of the patient;
generate, based on the one or more images, a representation of a plurality of potential anatomical states of the patient which may occur during the future radiation treatment session; and
generate, based on the representation, at least one treatment plan.

20. A non-transitory computer-readable medium for generating a radiation treatment plan for a patient for at least one future radiation treatment session, the non-transitory computer-readable medium comprising instructions which, when executed by a computer, cause the computer to:
obtain one or more images of the patient;
generate, based on the one or more images, a representation of a plurality of potential anatomical states of the patient which may occur during the future radiation treatment session; and
generate, based on the representation, at least one treatment plan.
